(19) United States
(12) Patent Application Publication
(10) Pub. No.: US 2008/0009801 A1
(43) Pub. Date: Jan. 10, 2008

(54) Method for dispensing material into a drug delivery device

(76) Inventor: Janice H. Nickel, Sunnyvale, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/001,587

(22) Filed: Dec. 2, 2004

(51) Int. Cl. A61M 5/00

(52) U.S. Cl. 604/173; 604/20; 604/47

(57) ABSTRACT

In a method for dispensing a material into a drug delivery device, a transdermal drug delivery device having reservoirs is provided, in which the reservoirs are in fluid communication with microneedles configured for insertion into a user's skin. Instructions to deposit first and second materials are received and a first set of reservoirs to receive the first material and a second set of reservoirs to receive the second material are selected. In addition, the first material is deposited into the first set of reservoirs and the second material is deposited into the second set of reservoirs through operation of a material dispensing device.

### CROSS-REFERENCE TO RELATED APPLICATION

This application is related to commonly assigned and co-pending U.S. patent application Ser. No. XX/XXX,XXX, (Attorney Docket No. 200405118-1) entitled “Transdermal Drug Delivery Device”, filed on even date herewith, the disclosure of which is hereby incorporated by reference in its entirety.

### BACKGROUND

Various techniques are known for delivering drugs into humans and animals. A more common set of these techniques include orally delivered drugs, such as pills or capsules, transdermally delivered drugs, such as, syringes or catheters, and transdermal patches. While typically effective for drug delivery, these techniques have certain drawbacks. For instance, the effectiveness of orally delivered drugs is often reduced due to degradation caused in the digestive system. The use of syringes or catheters typically require administration by a person trained in their use and are often associated with pain and local damage to the skin. Transdermal patches often have limited applicability due to the inability of larger molecules to penetrate the dermal layer.

Another, more recently developed technique for delivering drugs to users includes the use of devices having micro-machined needles. These devices are typically fabricated to include a very large number of microneedles configured to penetrate across the dermal barrier. Although these devices have been found to be effective in enabling relatively painless drug delivery, they do have some shortfalls. For instance, the types of drugs delivered through use of these devices are limited to those supplied in the devices during their manufacture. In other words, the drugs to be administered with known drug delivery devices of this type are integrally manufactured with the drug delivery devices. As such, the types of drugs contained in the known drug delivery devices cannot be configured to deliver additional or different types of drugs other than those the devices were initially manufactured to deliver.

Thus, if a person is required to receive different types of medication through use of these devices, that person would be required to obtain a plurality of these devices, track which of these devices contain the appropriate drugs, and make sure that all of these devices are properly attached to their skin to enable the drugs to be properly administered. This may prove difficult for certain people as they may forget to administer certain ones of the drugs.

Accordingly, it would be beneficial to have a more flexible drug delivery device capable of delivering a relatively wide variety of drugs, in particular, it would be beneficial to have a dispensing system capable of filling a drug delivery device with various drugs according to a user's customized needs.

### SUMMARY

A method for dispensing a material into a drug delivery device is disclosed herein. In the method, a transdermal drug delivery device having reservoirs is provided, in which the reservoirs are in fluid communication with microneedles configured for insertion into a user's skin. Instructions to deposit first and second materials are received and a first set of reservoirs to receive the first material and a second set of reservoirs to receive the second material are selected. In addition, the first material is deposited into the first set of reservoirs and the second material is deposited into the second set of reservoirs through operation of a material dispensing device.

### BRIEF DESCRIPTION OF THE DRAWINGS

Features of the present invention will become apparent to those skilled in the art from the following description with reference to the figures, in which:

FIG. 1A shows a simplified cross-sectional side view of a transdermal drug delivery device according to an embodiment of the invention;

FIG. 1B illustrates a simplified plan view of a cassette of the transdermal drug delivery device illustrated in FIG. 1A;

FIG. 2A illustrates a simplified schematic diagram of a material dispensing device for dispensing material into the reservoirs of cassettes, according to an embodiment of the invention;

FIG. 2B is a bottom view of a dispenser taken along lines II-Il in FIG. 2A;

FIG. 3 illustrates a block diagram of a control system for controlling a dispensing device, such as, the material dispensing device depicted in FIG. 2A, according to an embodiment of the invention;

FIG. 4A illustrates a flow diagram of an operational mode for depositing materials with a material dispensing device, according to an embodiment of the invention;

FIG. 4B illustrates a flow diagram of an operational mode for depositing materials with a material dispensing device, according to another embodiment of the invention; and

FIG. 5 illustrates a computer system, which may be employed to perform various functions described herein, according to an embodiment of the invention.

### DETAILED DESCRIPTION

For simplicity and illustrative purposes, the present invention is described by referring mainly to an exemplary embodiment thereof. In the following description, numerous specific details are set forth in order to provide a thorough understanding of the present invention. It will be apparent however, to one of ordinary skill in the art, that the present invention may be practiced without limitation to these specific details. In other instances, well known methods and structures have not been described in detail so as not to unnecessarily obscure the present invention.

As described in greater detail herein below, a transdermal drug delivery device includes a cassette portion and an array of microneedles, for instance, needles having lengths of about 1 μm to 1 mm. More particularly, the microneedles are sized and configured to deliver liquid materials contained in the drug delivery device through a dermal layer of skin. The cassette portion includes a number of reservoirs configured to individually hold the liquid materials. In addition, the reservoirs are formed such that liquid materials contained in one of the reservoirs may be kept separate from the liquid materials contained in others of the reservoirs. In this regard, a single transdermal drug delivery device may be used to store and deliver a number of different types of liquid materials, for instance, different types of drugs.

The transdermal drug delivery device may include a lid configured to assist in maintaining the separation among reservoirs. In addition, the lid may be removable, such that the reservoirs may be accessed when desired. In some instances, the lid may comprise the lid described in the U.S. patent application Ser. No. XX/XXX,XXX, (Attorney Docket No. 200405118-1) entitled “Transdermal Drug Delivery Device”. In any regard, the reservoirs may be accessed such that they may be supplied with the liquid material.

A dispensing device may be employed to supply the reservoirs with the liquid materials. The dispensing device may be configured to deliver the liquid materials in droplets having sizes that are sufficiently small to be accurately delivered into individual ones of the reservoirs. For instance, the dispensing device may be configured to form droplets having sizes that range from around 1 picoliter to around 1 microliter or more. The dispensing device may also include different types of liquid materials such that the different types of liquid materials may be delivered into the reservoirs. In addition, either or both of the dispensing device and the drug delivery device may be movable with respect to each other such that various ones of the reservoirs may be accessed by the dispensing device.

Through implementation of the various examples described herein, reservoirs formed in a transdermal drug delivery device may be supplied with precise amounts of liquid materials. In addition, the reservoirs may contain a variety of different types of liquid materials, such that, a single transdermal drug delivery device may be employed to administer a relatively large number of different types of liquid materials transdermally.

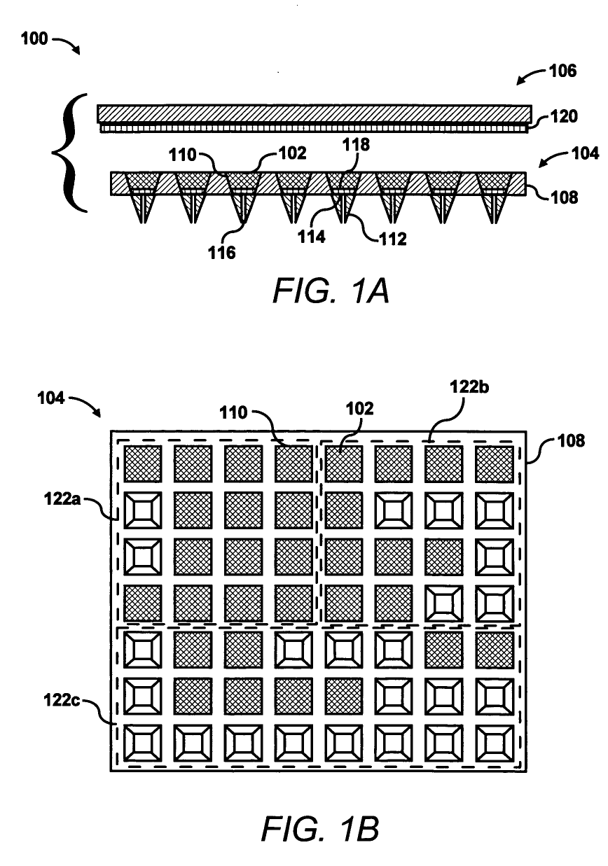

With reference to FIG. 1A, there is shown a simplified cross-sectional side view of a transdermal drug delivery device 100. It should be readily apparent that the transdermal drug delivery device 100 depicted in FIG. 1A represents a generalized illustration and that other elements may be added or existing elements may be removed or modified without departing from a scope of the transdermal drug delivery device 100. For example, the transdermal drug delivery device 100 may include additional layers, additional reservoirs and microneedles, etc.

The transdermal drug delivery device 100 is generally configured to receive and store drugs 102, such as, medicines, vaccines, or other agents known or heretofore known to be administered transdermally. The transdermal drug delivery device 100 is also configured to be placed on a user's skin such that the drug 102 contained in the device 100 may be delivered transdermally. In addition, the transdermal drug delivery device 100 may be configured to deliver the drug 102 as or when the transdermal drug delivery device 100 is placed on the user's skin. Alternatively, the transdermal drug delivery device 100 may be equipped with a mechanism designed to control a timing of drug 102 delivery into the user's skin. In this regard, the transdermal drug delivery device 100 may be equipped with adhesives or the like to enable the device 100 to be adhered to the user's skin.

As shown in FIG. 1A, the transdermal drug delivery device 100 includes a cassette 104 and a lid 106. The cassette 104 includes a substrate 108 having a plurality of reservoirs 110 formed throughout the substrate 108. The substrate 108 may be constructed from any reasonably suitable material. Suitable materials may include, for instance, silicon, metals, ceramics, polymers, composites and the like. In addition, the substrate 108 may be formed of flexible or rigid materials.

In addition, a plurality of microneedles 112 are formed on a lower surface of the substrate 108. The microneedles 112 are formed such that they are in fluid communication with one or more of the reservoirs 110 through respective openings 114. As shown in FIG. 1A, however, the microneedles 112 are each in fluid communication with a respective one of the reservoirs 110. In any respect, the microneedles 112 are sized and shaped to penetrate the stratum corneum layer of a user's skin. In addition, the microneedles 112 include channels 116 having sufficient diameters to permit passage of the drug 102 contained in the reservoirs 110 through the microneedles 112. In one example, the microneedles may have lengths ranging from about 1 μm to 1 mm. In addition, an array of 100 or more microneedles 112 may be formed on the substrate 108.

The openings 114 at the interfaces between the reservoirs 110 and the microneedles 112 may be covered with respective membranes 118. Examples of suitable materials for the membranes 118 comprise polymers, ceramics, metals, glasses, etc. The membranes 118 may be configured to provide a liquid seal of the reservoirs 110 and to substantially prevent contamination of the drugs 102 contained in the reservoirs 110. The membranes 118 may also be configured to open or otherwise enable the drugs 102 contained in the reservoirs 110 to flow through the openings 114 when desired. In one example, the membranes 118 may be configured to rupture when at least a predetermined amount of force is exerted on the drugs 102. In another example, the membranes 118 may comprise diffusive materials configured to control the rate at which the drugs 102 flow out of the openings 114. In any of these examples, the rates at which the drugs 102 are expelled from various reservoirs 110 may be controlled such that drugs 102 contained in different reservoirs 110 may be delivered at different times. For instance, certain of the reservoirs 110 may be equipped with membranes 118 configured to enable the drug 102 to pass there through at a first rate while others of the reservoirs 110 may be equipped with membranes 118 configured to enable the drug 102 to pass there through at a second rate, and so forth.

The cassette 104 and the lid 106 may be formed through any number of reasonably suitable manufacturing techniques. For instance, the cassette 104, including the reservoirs 110 and the microneedles 112, may be formed using standard MEMS (MicroElectro-Mechanical System) manufacturing techniques. In addition, the cassette 104 and the lid 106 may be formed using other methods known to those skilled in the art.

The lid 106 may be attached to the cassette 104 to provide a liquid seal of the drugs 102 contained in the reservoirs 110. In this regard, the lid 106 may be bonded to the cassette 104 through use of an adhesive 120. The adhesive 120 may, for instance, be pressure-activated, heat-activated, or the like. In addition, the adhesive 120 may be selected to provide an adequate seal at the interface between the lid 106 and the cassette 104. As an alternative to the use of adhesives, the lid 106 may be attached to the cassette 104 through other suitable means. For instance, the lid 106 or the cassette 104 may be formed of a material designed to be bonded to the cassette 104 through application of heat, light, or other types of energy. As another example, the lid 106 and the cassette 104 may be formed with complimentary structures configured to mate with one another and provide an interlocking connection between the lid 106 and the cassette 104.

In any respect, the lid 106 may be attached to the cassette 104 following insertion of the drugs 102 into the reservoirs 110. In addition, although the lid 106 is shown as being separate from the cassette 104, the lid 106 may be integrally formed with the cassette 104. In this instance, the lid 106 may be attached to the cassette 104 through use of a hinge (not shown) which enables access to the reservoirs 110. In a further example, the lid 106 may comprise the lid described in the U.S. patent application Ser. No. XX/XXX,XXX, (Attorney Docket No. 200405118-1) entitled “Transdermal Drug Delivery Device”. As also described in that application, some of the reservoirs 110 may be configured to house electrolyte materials for providing electrical energy to a number of electrical devices configured on the delivery device 100. As discussed herein below, a material dispensing device may be employed to fill these reservoirs with the electrolyte materials.

Turning now to FIG. 1B, there is shown a simplified plan view of the cassette 104 illustrated in FIG. 1A. The cassette 104 is shown in FIG. 1B as containing a particular configuration in which certain of the reservoirs 110 do not contain drugs 102. It should be understood that the number of reservoirs 110 that are empty as well as the number of reservoirs 110 depicted are not meant to limit the cassette 104 in any respect but have been so illustrated to provide a thorough understanding of a possible cassette 104 configuration.

As shown in FIG. 1B, a number of reservoirs 110 are positioned in an array on the cassette 104, such that, the cassette 104 may include a relatively large number of reservoirs 110. In addition, some of the reservoirs 110 are illustrated as being empty, which may indicate an instance where, for example, during a filling operation of the reservoir 110. The reservoirs 110 may be assigned to hold different types of drugs 102. For instance, the reservoirs 110 contained in the outlined section 122a may be configured to hold a first type of drug 102, the reservoirs 110 contained in the outlined section 122b may be configured to hold a second type of drug 102, and the reservoirs 110 contained in the outlined section 122c may be configured to house a third type of drug 102. In this regard, a single cassette 104 may be used to transdermally deliver any reasonably suitable number of drugs 102 to a user. In addition, since the timing of the deliveries of the various drugs 102 may be varied, the times or frequencies at which the various drugs 102 are delivered to a user may also be controlled. Thus, a user who is required to receive various medications at various times during a day, for instance, may do so through use of a single cassette 104.

By way of example, if a drug A contained in the outlined section 122a is configured to be delivered to the user two times a day, some of the reservoirs 110 may include membranes 118 designed to dissolve or otherwise enable passage of the drugs A contained therein at one part of the day. In addition, others of the reservoirs 110 may include membranes 118 designed to dissolve or otherwise enable passage of the drugs A contained therein at another part of the day. In this regard, a user may receive multiple doses of a single medication through application of a single transdermal drug delivery device 100. The user may also receive multiple doses of multiple medications through similar reservoir 110 and membrane 118 configurations.

The manners in which the reservoirs 110 are illustrated as being grouped into the outlined sections 122a-122c are for purposes of illustration only and are not intended to limit the transdermal drug delivery device 100 in any respect. In this regard, the reservoirs 110 may be assigned to hold any reasonably suitable number of drugs 102 in any reasonably suitable pattern.

In addition, although the transdermal drug delivery device 100 has been illustrated and described as having a membrane 118 that dissolves or otherwise enables passage of the drugs 102 out of the reservoirs 110 at prescribed times, it should be understood that the transdermal drug delivery device 100 may be configured to enable delivery of the drugs 102 through various other means. For instance, the membrane 118 may comprise a material configured to become ruptured or otherwise open through, for instance, application of force onto the transdermal drug delivery device 100 by a user. In this case, the transdermal drug delivery device 100 may be employed for the simultaneous administration of one or more drugs, for example, one or more vaccines.

As another example, the transdermal drug delivery device 100 may be equipped with means for applying pressure onto the drugs 102 to cause the drugs 102 to be expelled from their respective reservoirs 110. The means for applying pressure may include, for instance, a material configured to expand in response to predetermined temperatures, pH, or other environmental factors, such as, hydrogel actuators and the like. The means for applying pressure may also include an electrically activated actuator, such as, a pump actuation mechanism, a thermal inkjet element, a piezoelectric element, etc.

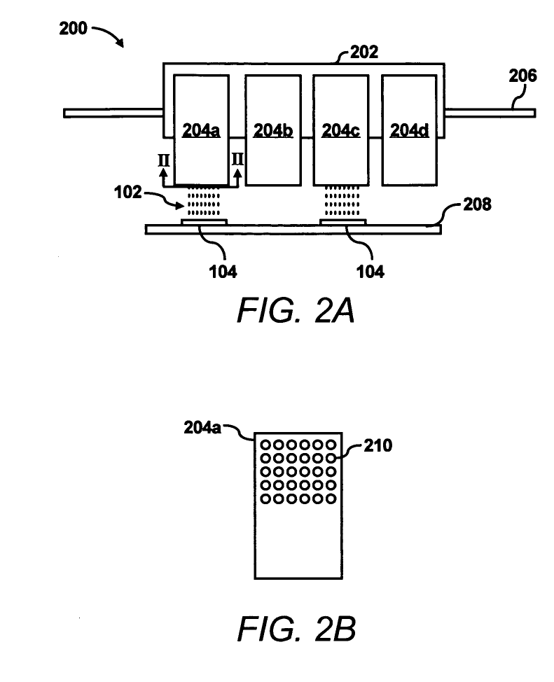

In any of the above-identified types of transdermal drug delivery devices 100, various types of drugs 102 may be deposited into various reservoirs 110 through use of a material dispensing device 200 (shown in FIG. 2A). In one respect, the various types of drugs 102 contained in the transdermal drug delivery devices 100 may be administered to a user at various prescribed times, such that, the user may receive a plurality of medications through use of a single transdermal drug delivery device 100.

FIG. 2A illustrates a simplified schematic diagram of a material dispensing device 200 for dispensing drugs 102 into the reservoirs 110 of cassettes 104. It should be readily apparent that the material dispensing device 200 depicted in FIG. 2A represents a generalized illustration and that other elements may be added or existing elements may be removed or modified without departing from a scope of the material dispensing device 200. For example, the material dispensing device 200 may include any number of dispensers configured to deposit at least one of the one or more drugs 102 and other materials onto transdermal drug delivery devices 100.

In FIGS. 2A and 2B, the material dispensing device 200 is illustrated as an inkjet delivery system. It should, however, be understood that the material dispensing device 200 may comprise other types of structures without departing from a scope of the material dispensing device 200. For instance, the material dispensing device 200 may include one or more mechanisms for accurately pumping the drugs 102 out through nozzles of the material dispensing device 200 and into the reservoirs 110. Thus, the inkjet delivery system configuration depicted in FIGS. 2A and 2B is illustrative of one example and the material dispensing device 200 may therefore include different configurations while remaining within a scope of the material dispensing device 200.

As shown in FIG. 2A, the material dispensing device 200 generally includes a carriage 202 configured to support a plurality of dispensers 204a-204d. In keeping with the example of the inkjet delivery system, the dispensers 204a-204d are illustrated in FIG. 2A as comprising inkjet printheads. The dispensers 204a-204d may, however, be configured with other dispensing means, such as, one or more pumping mechanisms.

In any regard, as will be described in greater detail herein below, the dispensers 204a-204d are configured to deposit at least one type of drug 102 into the reservoirs of a cassette 104. In addition, the relative positions of the dispensers 204a-204d and the cassette 104 may be controlled to thereby ensure that the drugs 102 are accurately deposited into desired ones of the reservoirs 110. In this regard, the positions of either or both of the dispensers 204a-204d and the cassette 104 may be varied to thus enable the at least one type of drug 102 to be deposited into the reservoirs 110.

The cassette 104 may be supported on a platform 208 of the material dispensing device 200. As shown, the platform 208 may simultaneously support a plurality of cassettes 104. In this regard, the dispensing device 200 may deliver the at least one drug 102 substantially simultaneously to a plurality of cassettes 104, thereby substantially reducing the time required to fill the cassettes 104 with the at least one drug 102. In certain instances, the platform 208 may be movable to thereby move the cassettes 104 with respect to the dispensers 204a-204d. In other instances, the support 208 may be stationary with respect to the dispensers 204a-204d.

In a first example, neither the dispensers 204a-204d nor the platform 208 may be movable with respect to each other. In this instance, the cassettes 104 may receive a single type of drug 102. Alternatively, the cassettes 104 may be sufficiently large such that they are capable of receiving different types of drug 102 from multiple dispensers 204a-204b. As a further alternative, the cassettes 104 may receive a first type of drug 102 from a first dispenser 204a, be moved to a location to receive a second type of drug 102 from a second dispenser 204b, and receive the second type of drug 102 from the second dispenser 204b. In this instance, an outside actuator, such as a robotic manipulator, a user, etc., may be employed to move the cassettes 104.

In a second example, the dispensers 204a-204d may be movable with respect to the cassette 104. In this example, the carriage 202 may be fixedly or movably supported on a guide member 206. If the carriage 202 is movably supported on the guide member 206, the position of the carriage 202 may be varied through relative movement between the carriage 202 and the guide member 206. Alternatively, if the carriage 202 is fixedly supported on the guide member 206, the position of the carriage 202 and therefore the dispensers 204a-204d may be varied through movement of the guide member 206. Thus, although not shown, one or more actuators, for instance, DC motors and the like, may be employed to move either or both of the carriage 202 and the guide member 206. In addition, the position of the carriage 202 and/or the guide member 206 may be detected through use of any reasonably suitable known position detection device (not shown). An example of a suitable position detection device is an encoder, which may broadly encompass a wide range of different types of encoders that may be employed without deviating from a scope of the material dispensing device 200.

In a third example, the platform 208 may be movable with respect to the dispensers 204a-204d, such that, the platform 208 may accurately position the cassettes 104 to receive at least one type of drug 102 from selected dispensers 204a-204d. In this instance, one or more actuators (not shown), for instance, DC motors and the like, may be employed to vary the position of the platform 208. In addition, the position of the platform 208 and therefore the cassettes 104 may be detected through use of any reasonably suitable known position detection device. An example of a suitable position detection device is an encoder, which may broadly encompass a wide range of different types of encoders that may be employed without deviating from a scope of the material dispensing device 200.

In a fourth example, both the carriage 202 and the platform 208 may be movable with respect to each other in any of the manners described in the previous examples.

As stated above, the dispensers 204a-204d may each deliver the same type of drug 102 or they may deliver multiple types of drugs 102. In certain instances, at least one of the dispensers 204a-204d may deliver substances other than a drug 102 to be delivered to a user. By way of example, at least one of the dispensers, for instance, the dispenser 204a, may deliver electrolyte materials for use in generating electrical energy for at least one electronic device of the delivery device 100. Thus, for example, electrolytes may be dispensed into some of the reservoirs 110 by the dispenser 204a, and some or all of the remaining dispensers 204b-204d may deposit one or more drugs 102 into the remaining reservoirs 110.

In addition, one of the dispensers, for instance, the dispenser 204d, may deposit a finishing layer on the drugs 102 deposited in the reservoirs 110. The finishing layer may comprise, for instance, a layer configured to seal the drugs 102 to thereby substantially prevent interspersion between the drugs 102 and contamination of the drugs 102. Alternatively, additional dispensers may be included in the material dispensing device 200 to perform deposition processes before or after deposition of the drug 102 into the reservoirs 110.

The dispensers 204a-204d may be removably attached to the carriage 202, such that, they may be replaced as the drugs 102 contained therein are depleted. The dispensers 204a-204d may also be replaced, for instance, in situations where different types of drugs 102 are to be deposited onto the cassette 104. By way of example, if a certain combination of drugs 102 are to be deposited onto the cassette 104, the dispensers 204a-204d may be arranged on the carriage 202 to deposit the predetermined combination of drugs 102. The dispensers 204a-204d may contain respective cartridges (not shown) to contain the drugs 102 prior to their deposition into the cassettes 104.

Alternatively, the dispensers 204a-204d may receive the drugs 102 through tubing (not shown) from separately situated drug supplies (not shown). Examples of suitable drugs 102 include, for instance, vitamins, cholesterol lowering drugs (statins), blood pressure drugs (aspirin), etc. In any respect, the dispensers 204a-204d, transdermal drug delivery device 100, cartridges or other material supplies, as well other components of the material dispensing device 200 may be housed in a substantially sterile environment to thereby reduce the risks of contamination of the transdermal drug delivery device 100 and the drug 102.

According to an example, the material dispensing device 200 may be operated by individuals with at least a certain level of training, for instance, pharmacists, doctors, and the like. In this regard, there is a higher likelihood that the transdermal drug delivery device 100 will be filled with the correct drugs 102, in their prescribed amounts, and configured to deliver the drugs 102 at the times they are prescribed to be administered to a user.

Although not specifically illustrated in FIG. 2A, the material dispensing device 200 may include a device for attaching the lid 106 to the cassette 104 after the drug 102 has been deposited into the reservoirs 110. As described hereinabove, the lid 106 may be attached to the cassette 104 in a variety of different manners. In any of these manners, the lid 106 is configured to be securely attached to the cassette 104 to substantially prevent the drug 102 from escaping and to substantially prevent contamination of the drug 102.

With reference to FIG. 2B, there is shown a bottom view of a dispenser 204a taken along lines II-II in FIG. 2A. As shown in FIG. 2B, the dispenser 204a, which may be representative of the other dispensers 204b-204d, includes a plurality of nozzles 210 through which the drug 102 is expelled from the dispenser 204a and deposited onto the cassette 104. Although the nozzles 210 have been illustrated as being in aligned rows and columns, the nozzles 210 may be arranged in a staggered configuration as is known to those skilled in the art. In addition, although a plurality of nozzles 210 have been illustrated in FIG. 2B, the dispenser 204a may include a single nozzle 210 without departing from a scope of the dispenser 204a.

Actuating devices (not shown) may be positioned behind each of the nozzles 210 to expel drugs 102 or other material out of the nozzles 210. The actuating devices are configured and operated to expel relatively controlled amounts of the drug 102 or other material. In a first example, the actuating device may comprise a piezoelectric actuating device or a thermal actuating device, which may be operated in manners consistent with manners generally known to those skilled in the art. In a second example, the drug 102 or other material may be expelled through the nozzles 210 through operation of a pump mechanism (not shown).

In any regard, the drug 102 or other material may be dispensed in relatively controlled amounts into the reservoirs 110 contained in the cassette 104. Therefore, relatively precise amounts of drug 102 or other material may be deposited into the reservoirs 110 through use of the material dispensing device 200.

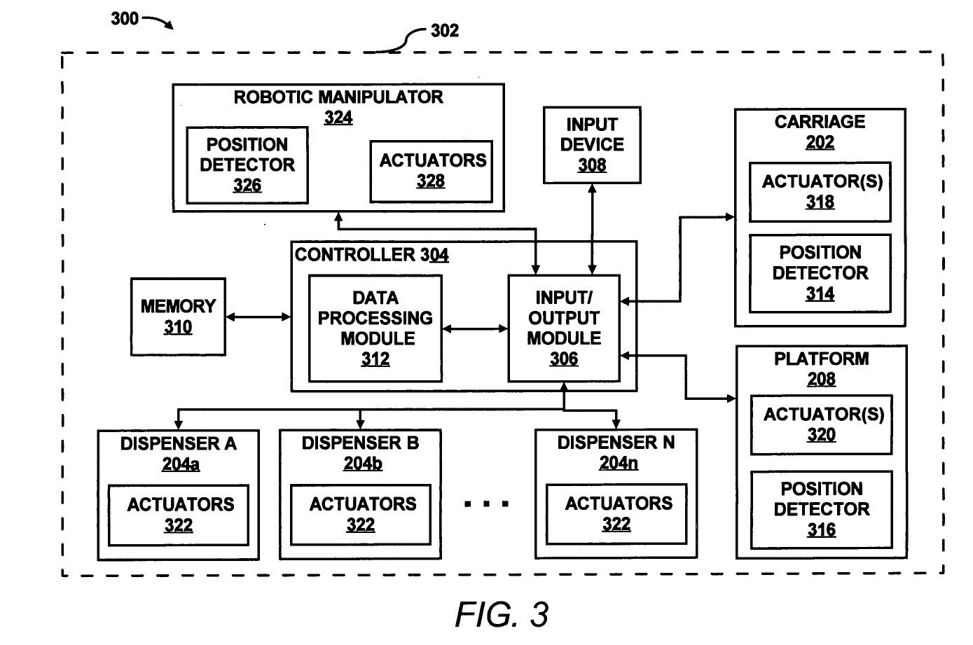

Various manners in which the dispensing device 200 may be operated will now be described with respect to FIG. 3. FIG. 3 depicts a block diagram 300 of a control system 302 for controlling the delivery system, such as, the dispensing device 200. It should be understood that the following description of the block diagram 300 is but one manner of a variety of different manners in which such a control system 302 may be operated to control operations of a material dispensing device 200. In addition, it should be understood that the control system 302 may include additional components and that some of the components described may be removed and/or modified without departing from a scope of the control system 302. Moreover, although particular reference is made to the material dispensing device 200 depicted in FIG. 2A, it should be understood that the control system 302 may be employed to control delivery systems having configurations that differ from that illustrated with respect to the material dispensing device 200.

The control system 302 includes a controller 304 configured to control various operations of the control system 302. The controller 304 may, for instance, comprise a computing device, a microprocessor, a micro-controller, an application specific integrated circuit (ASIC), and the like. In general, the controller 304 is configured to receive input, to process the data, and to control a material dispensing device 200 based upon the processed data as described in greater detail herein below.

The controller 304 includes an input/output module 306 configured to receive instructions as well as other information from various components of the material dispensing device 200. The input/output module 306 may thus function as an adapter for the controller 304 to receive and transmit data. In this regard, the input/output module 306 may comprise hardware and/or software configured to perform these functions. In addition, although the input/output module 306 has been illustrated as forming part of the controller 304, the input/output module 306 may comprise an algorithm stored in a memory 310 accessible by the controller 304. The memory 310 may also generally be configured to provide storage of software that provides the functionality of the controller 304. The memory 310 may be implemented, for instance, as a combination of volatile and non-volatile memory, such as DRAM, MRAM, EEPROM, flash memory, and the like.

An input device 308 may be used to input the instructions into the input/output module 306. The input device 308 may comprise, for instance, a user interface terminal, such as, a console on the material dispensing device 200. Alternatively, the input device 308 may comprise a computing device either attached or networked to the controller 304. In any regard, the instructions may include, for instance, the types of drugs 102 to be deposited onto a cassette 104, the amounts of drugs 102 to be deposited, the reservoirs 110 into which the drugs 102 are to be deposited, the prescribed timing at which the drugs 102 are to be administered to the user, etc.

The input/output module 306 may also be configured to receive data from various components contained in the material dispensing device 200. For example, the input/output module 306 may receive position information from one or both of the carriage 202 and the platform 208. More particularly, depending upon the configuration of the material dispensing device 200, either or both of the carriage 202 and the platform 208 may include respective position detectors 314, 316 configured to detect the respective positions of the carriage 202 and the platform 208. This position information may be transmitted or otherwise sent to the input/output module 306.

As another example, the input/output module 306 may receive information from the dispensers 204a-204n contained in the material dispensing device 200. This information may include, for instance, the types of drugs 102 contained in the respective dispensers 204a-204n, the levels of drugs 102 contained in the dispensers 204a-204n, the relative positions of the dispensers 204a-204n on the carriage 202, etc. This information may be transmitted or otherwise sent to the input/output module 306, for instance, when the dispensers 204a-204n are inserted into the carriage 202. The reference indicator “n” denotes any reasonably suitable number, such that, the control system 302 and the material dispensing device 200 may include may include any reasonably suitable number of dispensers 204a-204n.

The received instructions, position information, and dispenser 204a-204n information may be stored in the memory 310 for processing by a data processing module 312 of the controller 304. The data processing module 312 is generally configured to determine how the various components of the material dispensing device 200 are to be operated to perform the received instructions. The memory 310 may comprise software or algorithms that the data processing module 312 may implement in making these determinations. In addition, the processed data may be communication in the form of instructions through the input/output module 306 such that the instructions may be forwarded to the various components. In this regard the data processing module 312 may comprise hardware and/or software configured to perform these functions. Although the data processing module 312 has been shown in FIG. 3 as forming part of the controller 304, the functionality of the data processing module 312 may instead be stored in the form of a software or algorithm in the memory 310 without departing from a scope of the control system 302.

The controller 304 may transmit instructions to control an operation of at least one of the carriage 202, platform 208, and the dispensers 204a-204n. More particularly, for instance, the controller 304 may transmit instructions to control one or more actuators 318 configured to control operations of the carriage 202. By way of example, the actuator(s) 318 may be controlled to vary a position of the carriage 202 with respect to a cassette 104. As another example, the controller 304 may transmit instructions to control one or more actuators 320 for controlling operations of the platform 208. For instance, the actuator(s) 320 may be controlled to vary a position of the cassette 104 with respect to the dispensers 204a-204n. As another example, with respect to the dispensers 204a-204n, the controller 304 may control actuators 322 configured to control firing of the drug 102 out of the dispenser nozzles 210. In this regard, the actuators 322 may comprise piezoelectric actuating devices, thermal actuating devices, pump mechanism, etc.

The data processing module 312 may also be programmed to determine whether one or more drugs 102 to be dispensed into the delivery device 100 may be likely to cause an adverse reaction with one or more other drugs 102 or with another material, such as, a finishing material. If the data processing module 312 makes this determination, the data processing module 312 may provide an indication of the potential adverse reaction. In addition, or alternatively, the data processing module 312 may prevent the adversely reactive drugs 102 or other materials from being dispensed into the delivery device 100.

The control system 302 may include additional components that may assist in the deposition of drugs 102 into the reservoirs 110 of the cassette 104. An example of an additional component is a robotic manipulator 324 that may be employed by the control system 302 to perform various functions with respect to the cassette 104. For instance, the robotic manipulator 324 may be used to position the cassette 104 onto the substrate 208 with relatively greater precision than is possible through human positioning. As another example, the robotic manipulator 324 may be used to attach the lid 106 to the cassette 104 following deposition of the drug 102 into the cassette 104 as well as to remove the transdermal drug delivery device 100 from the platform 208 following completion of the drug 102 deposition process.

The robotic manipulator 324 may also be employed, for instance, to remove and/or replace dispensers 204a-204n. Thus, for example, if the controller 304 determines that none of the dispensers 204a-204n currently positioned on the carriage 202 contain the correct drug 102 to be deposited onto the cassette 104, the controller 304 may operate the robotic manipulator 324 to add the correct dispenser 204n and to remove an existing dispenser 204a, as necessary, for instance, to provide sufficient space for the additional dispenser 204n.

In performing any of these functions, the position of the robotic manipulator 324 may be tracked through use of a position detector 326 and the robotic manipulator 324 movements may be effectuated through operation of a plurality of actuators 328. It should be understood that the robotic manipulator 324 may be optional, for instance, in situations where the platform 208 includes guides for enabling accurate manual placement of the cassettes 104 or when the positions of the cassettes 104 may otherwise be detected with sufficient levels of accuracy.

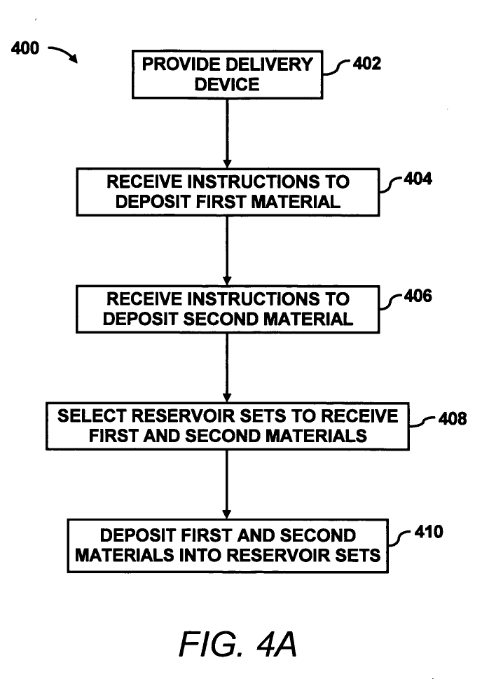

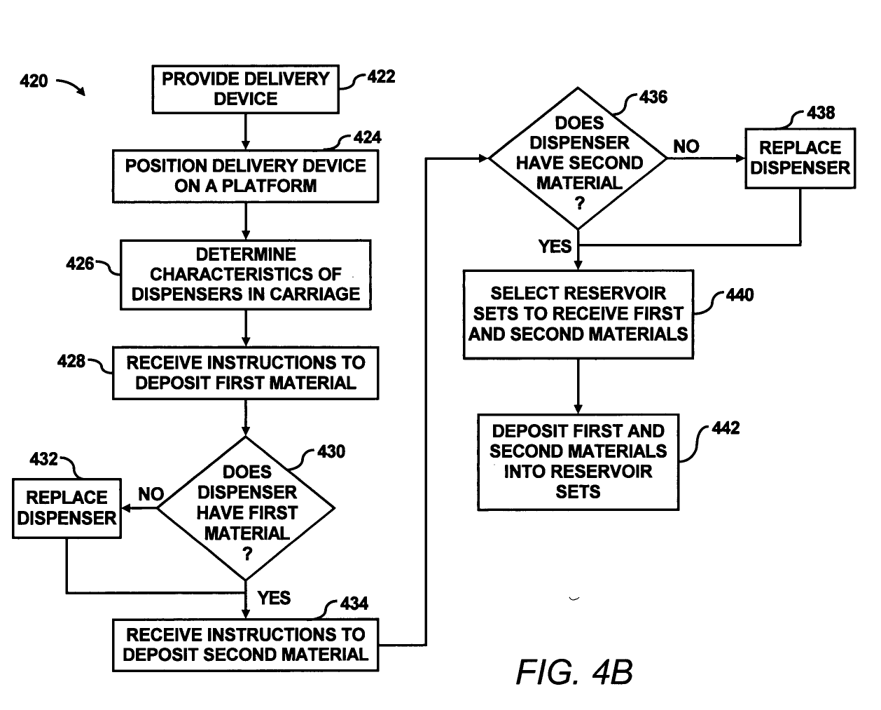

FIG. 4A illustrates a flow diagram of an operational mode 400 for depositing one or more drugs or other materials with a material dispensing device. It is to be understood that the following description of the operational mode 400 is but one manner of a variety of different manners in which the deposition of materials with a material dispensing device could be operated. It should also be apparent to those of ordinary skill in the art that the operational mode 400 represents a generalized illustration and that other steps may be added or existing steps may be removed or modified without departing from a scope of the operational mode 400. The description of the operational mode 400 is made with reference to the block diagram 300 illustrated in FIG. 3, and thus makes reference to the elements cited therein.

The operational mode 400 generally operates as an algorithm to deposit selected materials or drugs 102 into selected reservoirs in a transdermal drug delivery device 100. As shown in FIG. 4A, the transdermal drug delivery device 100 may be provided at step 402. Step 402 may also include the step of positioning the transdermal drug delivery device 100 on the platform 208 to receive drugs 102 and/or other materials from the dispensers 204a-204n. As described herein above, the material dispensing device 200 may be employed to dispense materials other than drugs 102 into the transdermal drug delivery device 100. The materials may include, for instance, electrolytes, substances useable to seal the drugs 102 in their respective reservoirs 110, etc.

At step 404, the controller 304 may receive instructions to deposit a first material, from, for instance, an input device 308. In addition, the controller 306 may receive instructions to deposit a second material, again, from the input device 308. These instructions may include the types of materials to be deposited into the transdermal drug delivery device 100 as well as their desired locations and amounts. These instructions may also include information pertaining to the desired timing at which, for instance, a first drug 102 and/or a second drug 102 are to be released from the transdermal drug delivery device 100.

Based upon the instructions received, the controller 304 may select in which of the reservoirs 110 formed in the cassette 104 of the transdermal drug delivery device 100 the first material and the second material are to be deposited at step 408. More particularly, for instance, the controller 304 may map the reservoirs 110 into various sections as shown in FIG. 1B. The selection of where to deposit the first material and the second material may be based upon, for instance, the desired times at which the first material and the second material are to be delivered by the transdermal drug delivery device 100. For instance, a first set of reservoirs 110 may include membranes 118 designed to enable drug 102 passage there through at a first time, whereas a second set of reservoirs 110 may include membranes 118 designed to enable drug 102 passage there through at a second time, and so forth. The controller 304 may be programmed with this information and may thus employ this information as a basis for determining into which of the reservoirs 110 the first and second drugs 102 are to be deposited.

As another example, the first set of reservoirs 110 may be designated to receive a drug 102 and the second set of reservoirs 110 may be designated to receive electrolyte materials. As above, the controller 304 may be programmed with this information and may thus employ this information to selectively dispense the drug 102 into the first set of reservoirs 110 and the electrolyte materials into the second set of reservoirs 110.

At step 410, the controller 304 may control the dispensers 204a-204n to deposit the first and second materials into the reservoir sets selected at step 408. Various manners in which the dispensers 204a-204n may be operated are described in greater detail hereinabove with respect to FIG. 3. Additional steps that may be performed in depositing materials with a material dispensing device are now described with respect to FIG. 4B.

FIG. 4B illustrates a flow diagram of an operational mode 420 for depositing materials with a material dispensing device. It is to be understood that the following description of the operational mode 420 is but one manner of a variety of different manners in which the deposition of materials with a material dispensing device could be operated. It should also be apparent to those of ordinary skill in the art that the operational mode 420 represents a generalized illustration and that other steps may be added or existing steps may be removed or modified without departing from a scope of the operational mode 420. The description of the operational mode 420 is made with reference to the block diagram 300 illustrated in FIG. 3, and thus makes reference to the elements cited therein.

In similar fashion to step 402 in FIG. 4A, the transdermal drug delivery device 100 may be provided at step 422. In addition, the transdermal delivery device 100 or the cassette 104 may be positioned on the platform 208 to receive drugs 102 or other materials from the dispensers 204a-204n at step 424.

At step 426, one or more characteristics of the dispensers 204a-204n supported on the carriage 202 may be determined. The one or more characteristics of the dispensers 204a-204n may include the types of materials contained in the dispensers 204a-204n, the amounts of materials contained in the dispensers 204a-204n, service records of the dispensers 204a-204n, etc. In addition, the controller 304 may receive instructions to deposit a first material at step 428 and the controller 304 may determine based upon the information obtained at step 426 as to whether any of the dispeners 204a-204n contain the first material at step 430. If the controller 304 determines that none of the dispensers 204a-204n contains the first material or is otherwise configured to deposit the first material, at least one of the dispensers 204a-204n may be replaced as indicated at step 432. More particularly, at least one of the dispensers 204a-204n may be replaced with one or more dispensers 204a-204n that contain the first material or are otherwise configured to deposit the first material at step 432.

Following either steps 430 or 432, the controller 304 may receive instructions to deposit a second material at step 434. The instructions to deposit the second material may also have been received at step 428 without deviating from a scope of the operational mode 420. In any regard, the controller 304 may determine based upon the information obtained at step 426 as to whether any of the dispensers 204a-204n contain the second material at step 436. If the controller 304 determines that none of the dispensers 204a-204n contains the second material or is otherwise configured to deposit the second material, at least one of the dispensers 204a-204n may be replaced as indicated at step 438. More particularly, at least one of the dispensers 204a-204n may be replaced with one or more dispensers 204a-204n that contain the second material or are otherwise configured to deposit the first material at step 438.

The dispenser 204a-204n replacement steps 432 and 438 may be performed manually by a user or the dispensers 204a-204n may be replaced automatically. In the event the control system 302 is configured with a robotic manipulator 324, the controller 304 may control the robotic manipulator 324 to perform the removal and replacement operations as different types of materials are required or when material levels in the dispensers 204a-204n fall below a predetermined level.

In any regard, following either of steps 436 and 438, the controller 304 may select in which of the reservoirs 110 formed in the cassette 104 of the transdermal drug delivery device 100 the first material and the second material are to be deposited at step 440. The selection of which of the reservoirs 110 are to receive which of the materials may be based, for instance, upon the instructions received at steps 426 and 434. More particularly, for instance, the controller 304 may map the reservoirs 110 into various sections as shown in FIG. 1B. The selection of where to deposit the first material and the second material may be based upon, for instance, the desired times at which a first drug 102 and a second drug 102 are to be delivered by the transdermal drug delivery device 100. In a first example, the controller 304 may employ this selection process in instances where a first set of reservoirs 110 includes membranes 118 designed to enable drug 102 passage there through at a first time and a second set of reservoirs 110 include membranes 118 designed to enable drug 102 passage there through at a second time, and so forth. The controller 304 may be programmed with this information and may thus employ this information as a basis for determining into which of the reservoirs 110 the first and second drugs 102 are to be deposited. The controller 304 may also control the dispensers 204a-204n to dispense the first and second materials into the selected sets of reservoirs 110 at step 442.

In another example, the controller 304 may be programmed to note that a first set of reservoirs 110 is designated to receive a drug 102 and that the second set of reservoirs 110 is designated to receive electrolyte materials. In addition, the controller 304 may employ this information to selectively dispense the drug 102 into the first set of reservoirs 110 and the electrolyte materials into the second set of reservoirs 110, at step 442.

Through implementation of the operational modes 400 and 420, transdermal drug delivery devices 100 may be supplied with customized materials (or drugs). In addition, the times at which the drugs 102 are delivered by the transdermal drug delivery devices 100 may be controlled. Thus, in one respect, a user may use a single transdermal drug delivery device 100 to receive at least one drug at various times.

The operations illustrated in the operational modes 400 and 420 may be contained as a utility, program, or a subprogram, in any desired computer accessible medium. In addition, the operational modes 400 and 420 may be embodied by a computer program, which can exist in a variety of forms both active and inactive. For example, they can exist as software program(s) comprised of program instructions in source code, object code, executable code or other formats. Any of the above can be embodied on a computer readable medium, which include storage devices and signals, in compressed or uncompressed form.

Exemplary computer readable storage devices include conventional computer system RAM, ROM, EPROM, EEPROM, and magnetic or optical disks or tapes. Exemplary computer readable signals, whether modulated using a carrier or not, are signals that a computer system hosting or running the computer program can be configured to access, including signals downloaded through the Internet or other networks. Concrete examples of the foregoing include distribution of the programs on a CD ROM or via Internet download. In a sense, the Internet itself, as an abstract entity, is a computer readable medium. The same is true of computer networks in general. It is therefore to be understood that any electronic device capable of executing the above-described functions may perform those functions enumerated above.

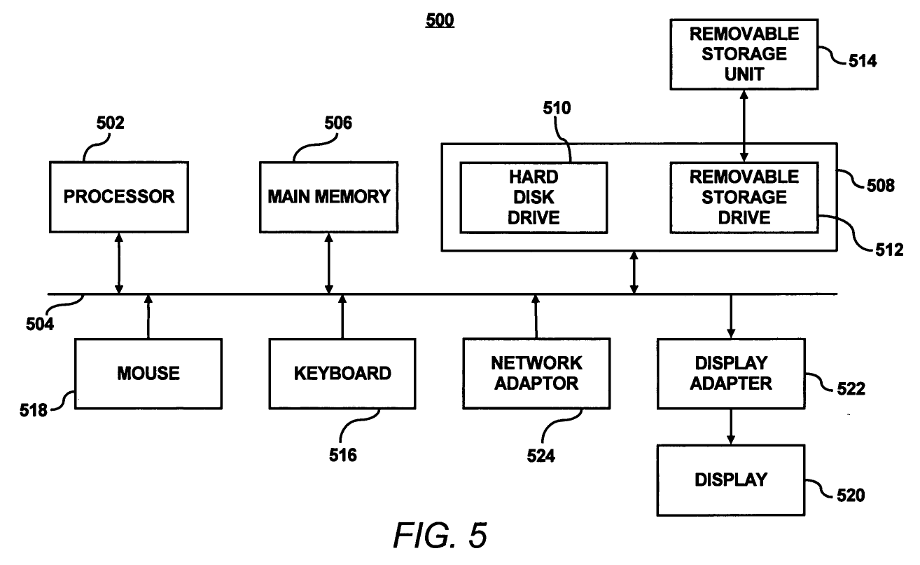

FIG. 5 illustrates a computer system 500, which may be employed to perform various functions described herein. The computer system 500 may include, for example, the controller 304 and/or the input device 308. In this respect, the computer system 500 may be used as a platform for executing one or more of the functions described herein above with respect to the various components of the control system 302.

The computer system 500 includes one or more controllers, such as a processor 502. The processor 502 may be used to execute some or all of the steps described in the operational modes 400 and 420. Commands and data from the processor 502 are communicated over a communication bus 504. The computer system 500 also includes a main memory 506, such as a random access memory (RAM), where the program code for, for instance, the controller 304 and/or the input device 308, may be executed during runtime, and a secondary memory 508. The secondary memory 508 includes, for example, one or more hard disk drives 510 and/or a removable storage drive 512, representing a floppy diskette drive, a magnetic tape drive, a compact disk drive, etc., where a copy of the program code for the control system 302 may be stored.

The removable storage drive 510 reads from and/or writes to a removable storage unit 514 in a well-known manner. User input and output devices may include a keyboard 516, a mouse 518, and a display 520. A display adaptor 522 may interface with the communication bus 504 and the display 520 and may receive display data from the processor 502 and convert the display data into display commands for the display 520. In addition, the processor 502 may communicate over a network, for instance, the Internet, LAN, etc., through a network adaptor 524.

It will be apparent to one of ordinary skill in the art that other known electronic components may be added or substituted in the computer system 500. In addition, the computer system 500 may include a system board or blade used in a rack in a data center, a conventional “white box” server or computing device, etc. Also, one or more of the components in FIG. 5 may be optional (for instance, user input devices, secondary memory, etc.).

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention, which is intended to be defined by the following claims—and their equivalents—in which all terms are meant in their broadest reasonable sense unless otherwise indicated.

What is claimed is:

1. A method for dispensing a material into a drug delivery device, said method comprising:
providing a drug delivery device having reservoirs, said reservoirs being in fluid communication with microneedles configured for insertion into a user's skin;
receiving instructions to deposit a first material;
receiving instructions to deposit a second material;
selecting a first set of reservoirs to receive the first material and a second set of reservoirs to receive the second material; and
depositing the first material into the first set of reservoirs and the second material into the second set of reservoirs through operation of a material dispensing device.

2. The method according to claim 1, wherein the material dispensing device comprises an inkjet delivery system having a plurality of printheads, a first one of the plurality of printheads housing the first material and a second one of the plurality of printheads housing the second material, wherein the step of depositing the first material comprises depositing the first material with the first printhead and wherein the step of depositing the second material comprises depositing the second material with the second printhead.

3. The method according to claim 1, wherein the material dispensing device comprises a plurality of dispensers supported on a carriage, said method further comprising:
determining whether the plurality of dispensers comprises the first material;
selecting another dispenser comprising the first material in response to the plurality of dispensers not comprising the first material; and
placing the another dispenser on the carriage, and wherein the step of depositing the first material comprises depositing the first material with the another dispenser.

4. The method according to claim 3, further comprising:
employing a robotic manipulator to select and to place the another dispenser on the carriage.

5. The method according to claim 1, further comprising:
selecting an amount of the first material to be deposited into the first set of reservoirs;
selecting an amount of the second material to be deposited into the second set of reservoirs;
depositing the selected amount of first material into the first set of reservoirs; and
depositing the selected amount of the second material into the second set of reservoirs.

6. The method according to claim 1, further comprising:
providing at least one membrane in the plurality of reservoirs, wherein the membrane controls delivery of the first material from the first set of reservoirs and delivery of the second material from the second set of reservoirs.

7. The method according to claim 6, wherein the step of providing at least one membrane comprises providing a membrane having a first height in the first set of reservoirs and providing a membrane having a second height in the second set of reservoirs to thereby cause the material in the first set of reservoirs to be delivered at a first time and the material in the second set of reservoirs to be delivered at a second time.

8. The method according to claim 1, wherein the first material and the second material comprises a drug and wherein the step of selecting a first set of reservoirs and a second set of reservoirs further comprises selecting a first set of reservoirs configured to release the drug at a first time and selecting a second set of reservoirs configured to release the drug at a second time.

9. The method according to claim 1, wherein the first material comprises a first drug and the second material comprises a second drug and wherein the step of selecting a first set of reservoirs and a second set of reservoirs further comprises selecting a first set of reservoirs configured to release the first drug at a first time and selecting a second set of reservoirs configured to release the second drug at a second time.

10. The method according to claim 1, wherein the first material comprises a drug and wherein the second material comprises an electrolyte material.

11. The method according to claim 1, further comprising:
determining whether the first material is potentially adversely reactive with the second material; and
providing an indication of the adverse reaction potential between the first material and the second material in response to the first material being potentially adversely reactive with the second material.

12. The method according to claim 1, further comprising:
placing a lid on the drug delivery device following the step of depositing the first material and the second material.

13. A transdermal drug delivery device comprising:
a cassette containing reservoirs for housing a first drug and a second drug, wherein a first set of reservoirs houses the first drug and the second set of reservoirs houses the second drug, and wherein the first drug is deposited into the first set of reservoirs and the second drug is deposited into the second set of reservoirs by a material dispensing device;
an array of microneedles formed on the cassette, wherein the microneedles are in fluid communication with respective ones of the reservoirs, and wherein the microneedles are configured for insertion into a user's skin and for conveying the first drug and the second drug into the user's skin; and
a lid for covering the reservoirs following deposition of the first and second drugs, wherein the lid is removable such that the first and second drugs are selected from a number of different types of drugs to thereby provide a transdermal drugs delivery device with customized drugs.

14. The transdermal drug delivery device according to claim 13, wherein material dispensing device comprises a first dispenser and a second dispenser, and wherein the first dispenser and the second dispenser each comprises at least one of piezoelectric actuators, thermal actuators, and a pumping mechanism for jetting the respective first drug and the second drug through the first dispenser and the second dispenser.

15. The transdermal drug delivery device according to claim 13, wherein an interface exists between the reservoirs and their respective microneedles, the transdermal drug delivery device further comprising:
a membrane positioned at each of the interfaces, said membrane being configured to control the migration of the first and second drugs from the reservoirs into the microneedles.

16. The transdermal drug delivery device according to claim 15, further comprising:
a first set of membranes configured to enable migration of the first drug from the first set of reservoirs at a first time; and
a second set of membranes configured to enable migration of the second drug from the second set of reservoirs at a second time, wherein the first time and the second time differ from each other.

17. A system for dispensing materials into a transdermal drug delivery device, said transdermal drug delivery device having reservoirs and microneedles, said reservoirs being in fluid communication with the microneedles through respective interfaces, said system comprising:
a carriage supporting a first dispenser and a second dispenser, wherein the first dispenser is configured to deposit a first material into a first set of reservoirs and wherein the second dispenser is configured to deposit a second material into a second set of reservoirs;
a platform supporting the transdermal drug delivery device at a location with respect to at least one of the first dispenser and the second dispenser to thereby enable the first material and the second material to be deposited into the respective reservoirs; and
a controller configured to control at least one of the first dispenser, the second dispenser, the carriage, and the platform to deposit the first and second materials into the respective reservoirs according to a predetermined layout of the first and second materials.

18. The system according to claim 17, further comprising:
an actuator for controlling the position of at least one of the carriage and the platform with respect to each other, wherein the controller is configured to control the actuator to thereby control the relative positions of the first and second dispensers and the first and second sets of reservoirs.

19. The system according to claim 17, wherein the first and second dispensers comprise nozzles, the system further comprising:
a plurality of actuators for ejecting the first material from the nozzles of the first dispenser and the second material from the second dispenser, wherein the plurality of actuators comprise at least one of piezoelectric actuators, thermal actuators, and pumping mechanisms, and wherein the controller is configured to control the plurality of actuators to thereby control deposition of the first and second materials into the first and second sets of reservoirs.

20. The system according to claim 17, further comprising:
a robotic manipulator for manipulating the transdermal drug delivery device, said robotic manipulator having an actuator, wherein the controller is configured to control the actuator to thereby vary operations of the robotic manipulator.

21. The system according to claim 17, further comprising:
a robotic manipulator for varying one or more dispensers supported on the carriage, wherein the controller is configured to control the robotic manipulator to thereby control the types of materials deposited from the one or more dispensers.

22. The system according to claim 17, wherein the first material comprises a drug and the second material comprises an electrolyte material.

23. The system according to claim 17, further comprising:
a third dispenser configured to deposit a material into the first and second sets of reservoirs following deposition of the first drug and the second drug, wherein the material comprises a layer configured to seal the first drug and the second drug within their respective reservoirs.

24. The system according to claim 17, wherein the first dispenser and the second dispenser are removable from the carriage, and wherein the carriage is configured to support at least one additional dispenser.

25. The system according to claim 17, wherein the first dispenser comprises a first inkjet printhead and the second dispenser comprises a second inkjet printhead.

26. A system comprising:
means for delivering at least one of a first material and a second material transdermally, said means for delivering comprising a plurality of reservoirs in fluid communication with an array of microneedles;
means for receiving instructions to deposit the first material and the second material;
means for depositing the first material and the second material into the plurality of reservoirs;
means for controlling the means for depositing the first and second materials to deposit the first material into a first set of reservoirs and to deposit the second material into a second set of reservoirs; and
means for controlling a timing of delivery of at least one of the first material and the second material, such that at least one of the first material and the second material is delivered at different times.

27. The system according to claim 26, further comprising:
means for automatically manipulating at least one of the means for delivering and the means for depositing.

28. A computer readable storage medium on which is embedded one or more computer programs, said one or more computer programs implementing a method for dispensing a first material and a second material into a transdermal drug delivery device having reservoirs with a material dispensing device, said one or more computer programs comprising a set of instructions for:
receiving instructions to deposit the first material;
receiving instructions to deposit the second material;
selecting a first set of reservoirs to receive the first material and a second set of reservoirs to receive the second material; and
depositing the first material into the first set of reservoirs and the second material into the second set of reservoirs through operation of the material dispensing device.

29. The computer readable storage medium according to claim 28, said one or more computer programs further comprising a set of instructions for:
determining whether a dispenser of the material dispensing device comprises the first material;
selecting another dispenser comprising the first material in response to the dispenser not comprising the first material; and
depositing the first material with the another dispenser.
